(19) United States
(12) Patent Application Publication
(10) Pub. No.: US 2015/0322515 A1
(43) Pub. Date: Nov. 12, 2015

(54) METHODS AND COMPOSITIONS FOR DETECTING TARGET SNP

(71) Applicant: NINGBO HEALTH GENE TECHNOLOGIES CO., LTD., Ningbo, Zhejiang (CN)

(72) Inventors: Linan WU, Ningbo, Zhejiang (CN); Jin YAN, Ningbo, Zhejiang (CN); Qingqing WANG, Ningbo, Zhejiang (CN); Yong WU, Ningbo, Zhejiang (CN)

(21) Appl. No.: 14/431,998

(22) PCT Filed: Jan. 3, 2014
(86) PCT No.: PCT/CN2014/070073

§ 371 Date: Mar. 27, 2015

(30) Foreign Application Priority Data
Jan. 25, 2013 (CN) 201310033261.9

(51) Int. Cl. C12Q 1/68

(52) U.S. Cl. CPC C12Q 1/6883; C12Q 2600/156

(57) ABSTRACT

The present invention provides methods and compositions, and uses thereof, for simultaneously detecting one target SNP locus or multiple target SNP loci in a sample. In exemplary embodiments, the present invention also provides a multiplex SNP assay technique, which can simultaneously detect up to 20 SNP loci (40 alleles) with high level of specificity (e.g., >99.9%), sensitivity (e.g. 100%) and accuracy, high throughput, cost-effective and time-saving, reduced or no false-negative results. The present invention further provides certain isolated polynucleotides that can be used as primers or primer pairs in the present methods and composition for simultaneously detecting one target SNP locus or multiple target SNP loci in a sample.

### I. CROSS-REFERENCE TO RELATED APPLICATION

This application claims the priority benefit of Chinese patent application No. 201310033261.9, filed Jan. 25, 2013, the content of which is incorporated by reference in its entirety.

### II. TECHNICAL FIELD

The present invention relates to methods and compositions, and uses thereof, for simultaneously detecting one SNP locus or multiple target SNP loci in a sample. In exemplary embodiments, the present invention also relates to a multiplex SNP assay technique, which can simultaneously detect up to 20 SNP loci (40 alleles) with high level of specificity (e.g., >99.9%), sensitivity (e.g., 100%) and accuracy, high-throughput, cost-effectiveness and time-saving, reduced or no false-negative results. The present invention further relates to certain isolated polynucleotides that can be used as primers or primer pairs in the present methods and compositions for simultaneously detecting one SNP locus or multiple target SNP loci in a sample.

### III. BACKGROUND OF THE INVENTION

Currently, studies found that many SNP loci are closely related with drug therapy, e.g., 8 SNP loci of 7 genes are related with 5-FU toxicity/ADR and efficacy including DPD rs3918290, DPD rs1801265, GSTP1 rs1695, MTHFR rs1801133, OPRT rs1801019, TYMS rs37473033, NOS3 rs1799983 and ERCC2 rs13181. Among those SNPs, DPD rs3918290 and TYMS rs37473033 have been confirmed by FDA and are suggested by FDA to do genotyping before 5-FU treatment. Therefore, it is necessary to develop a multiple SNP detection technique so that doctors can quickly provide drug toxicity/ADR and efficacy information to patients for safer and more efficient treatment. So far, there is a need for highly sensitive, highly accurate and low-cost multiple SNP detection methods in the art.

At present, the main methods to detect SNPs are DNA chip, Sanger sequencing and quantitative real-time PCR (qPCR). Some of their advantages and disadvantages for SNP detection are as follows:

### 1. DNA Microarray (DNA Chip)

A DNA microarray is a collection of microscopic DNA spots attached to a solid surface. Each DNA spot contains millions picomoles of a specific DNA sequence, known as probes. These can be a short section of a gene or other DNA element used to hybridize marked biological samples. Probe-target hybridization is usually detected and quantified by detection of fluorophore-, silver-, or chemiluminescence-labeled targets to determine relative abundance of nucleic acid sequences in the target.

The advantage of DNA chip is high-throughput SNP assay.

The disadvantages of DNA chip are:

1) Due to the different molecule thermodynamics between SNPs, it is difficult to control the conditions on detecting more SNPs in one condition.

2) The DNA chip technology is complicated. It is difficult to do probe synthesis, fixation, and make high density probe array.

3) DNA chip is expensive: one chip per sample, costing more than ¥1000/sample. That is not conducive to large-scale promotion.

4) Poor repeatability and accuracy: DNA chip is prone to obtain false positive result.

5) Low-sensitivity: DNA chip technique requires a relative large amount of nucleic acid. Usually, multiplex PCR amplification has to be done before SNP assay. Since the primers produce dimmers or hairpins easily, or the Tm value of primers is different, the DNA fragments are amplified with different efficiencies, thereby affecting detection sensitivity.

6) It's hard to establish quality standard for a great variety among DNA chips.

### 2. Sanger Sequencing Technique

Sanger sequencing is based on the selective incorporation of chain-terminating dideoxynucleotides by DNA polymerase during in vitro DNA replication. It is the gold standard of SNP analysis for the well-accepted accuracy. Sanger sequencing can detect known SNPs also unknown SNPs. The disadvantages of the technique are:
1) Complicated procedure with heavy workload. Several steps have to be done for each SNP locus: does PCR amplification, run an agarose gel, purify the DNAs in the gel, and then do sequencing.
2) Long cycle: 24 hours are needed for one reaction.
3) High cost: the cumulative price of multiple SNP loci detection is relatively expensive.
   3. Quantitative Real-Time PCR (qPCR) Method

Using fluorescence quenching technique and specific probes and primers, qPCR can detect SNP loci. Its advantage is high-sensitivity and high-accuracy. The disadvantages of the technique are:
1) Low-throughput: qPCR can only detect one target gene per reaction, so that it needs two reactions to finish a SNP detection (SNP and the corresponding allele).
2) It's impossible for qPCR to detect many SNP loci simultaneously.
3) It's impossible for qPCR to setup internal controls.
4) Expensive: two probes are needed for qPCR to detect a SNP allele, and If detection of several SNPs is need, the probes are very expensive.

In brief, the three techniques described above cannot meet the demand for rapid, accurate detection of a target SNP or multiple target SNPs. The present invention addresses this and other related needs in the field.

### IV. DISCLOSURE OF THE INVENTION

In one aspect, the present disclosure provides for a method for simultaneously detecting one target SNP locus or multiple target SNP loci in a sample, which method comprises: conducting multiplex PCR using a target polynucleotide in a sample as a template and multiple pairs of primers for one target SNP locus or multiple target SNP loci, and analyzing multiple PCR products using capillary electrophoresis, wherein said primers are designed so that the lengths of said PCR products from different SNP loci or from different alleles of the same SNP locus are sufficiently distinguishable from each other in capillary electrophoresis analysis.

In another aspect, the present disclosure provides for a kit or system for simultaneously detecting one target SNP locus or multiple target SNP loci in a sample, which kit or system comprises: 1) multiple pairs of primers for one target SNP locus or multiple target SNP loci; 2) means for conducting multiplex PCR using a target polynucleotide in a sample as a template and said multiple pairs of primers; and 3) means for analyzing multiple PCR products using capillary electrophoresis, wherein said primers are designed so that the lengths of said PCR products from different SNP loci or from different alleles of the same SNP locus are sufficiently distinguishable from each other in capillary electrophoresis analysis.

In some embodiments, the present disclosure provides for a multiplex SNP assay technique, which can simultaneously detect up to 20 SNP loci (40 alleles) with high level of specificity (e.g., >99.9%), sensitivity (e.g., 100%) and accuracy, high-throughput, cost-effective and time-saving, reduced or no false-negative results.

In still another aspect, the present disclosure provides for an isolated polynucleotide which comprises a polynucleotide sequence that exhibits at least 70%, 75%, 80%, 85%, 90%, 95%, 99% or 100% identity to any of the ERCC2, DPYD, GSTP1, MTHFR, OPRT, NOS3, DYPD, TS and pcDNA3.1(+) polynucleotide sequences set forth in Table 5, wherein said polynucleotide does not comprise a wild-type, full length ERCC2, DPYD, GSTP1, MTHFR, OPRT, NOS3, DYPD, TS and pcDNA3.1(+) polynucleotide sequence from which said polynucleotide is derived.

In yet another aspect, the present disclosure provides for a primer composition, which primer composition comprises, consists essentially of or consists of any of the primer pairs set forth in Table 5.

### V. BRIEF DESCRIPTION OF THE DRAWINGS

FIG. 1 illustrates an exemplary electropherogram of 5-FU panel assay on a patient blood sample. The electropherogram shows the analysis result of the 5-FU panel using a patient blood sample as template. The 5-FU panel is able to simultaneously analyze 8 SNP genotypes associated with the toxicity/ADR, efficacy and prognosis of 5-FU and a reference (internal PCR control) gene. The alleles of the patent are: ERCC2 AA, DYPD CC (rs3918290), GSTP1 GA, NOS3 GG, TS 3 repeats, MTHFR AA, OPRT GC, and DYPD TT (rs1801265).

### VI. DETAILED DESCRIPTION OF THE INVENTION

### A. Definitions

Unless defined otherwise, all technical and scientific terms used herein have the same meaning as is commonly understood by one of ordinary skill in the art to which this invention belongs. All patents, patent applications (published or unpublished), and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “mammal” refers to any of the mammalian class of species. Frequently, the term “mammal,” as used herein, refers to humans, human subjects or human patients.

As used herein, the term “subject” is not limited to a specific species or sample type. For example, the term “subject” may refer to a patient, and frequently a human patient. However, this term is not limited to humans and thus encompasses a variety of mammalian species.

As used herein the term “sample” refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

The terms “polynucleotide,” “oligonucleotide,” “nucleic acid” and “nucleic acid molecule” are used interchangeably herein to refer to a polymeric form of nucleotides of any length, e.g., at least 8, 9, 10, 20, 30, 40, 50, 100, 200, 300, 400, 500, 1,000 or more nucleotides, and may comprise ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), including tRNA, rRNA, hRNA, and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids (“PNAs”)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. Thus, these terms include, for example, 3′-deoxy-2′,5′-DNA, oligodeoxyribonucleotide N3′ to P5′ phosphoramidates, 2′-O-alkyl-substituted RNA, hybrids between DNA and RNA or between PNAs and DNA or RNA, and also include known types of modifications, for example, labels, alkylation, “caps,” substitution of one or more of the nucleotides with an analog, intemucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including enzymes (e.g. nucleases), toxins, antibodies, signal peptides, poly-L-lysine, etc.), those with intercalators (e.g., acridine, psoralen, etc.), those containing chelates (of, e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

It will be appreciated that, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides can also include modifications on the sugar moiety, e.g., wherein one or more of the hydroxyl groups are replaced with halogen, aliphatic groups, or are functionalized as ethers, amines, or the like. The term “nucleotidic unit” is intended to encompass nucleosides and nucleotides.

“Nucleic acid probe” and “probe” are used interchangeably and refer to a structure comprising a polynucleotide, as defined above, that contains a nucleic acid sequence that can bind to a corresponding target. The polynucleotide regions of probes may be composed of DNA, and/or RNA, and/or synthetic nucleotide analogs.

As used herein, “complementary or matched” means that two nucleic acid sequences have at least 50% sequence identity. Preferably, the two nucleic acid sequences have at least 60%, 70%, 80%, 90%, 95%, 96%, 97%, 98%, 99% or 100% of sequence identity. “Complementary or matched” also means that two nucleic acid sequences can hybridize under low, middle and/or high stringency condition(s).

As used herein, “substantially complementary or substantially matched” means that two nucleic acid sequences have at least 90% sequence identity. Preferably, the two nucleic acid sequences have at least 95%, 96%, 97%, 98%, 99% or 100% of sequence identity. Alternatively, “substantially complementary or substantially matched” means that two nucleic acid sequences can hybridize under high stringency condition(s).

In general, the stability of a hybrid is a function of the ion concentration and temperature. Typically, a hybridization reaction is performed under conditions of lower stringency, followed by washes of varying, but higher, stringency. Moderately stringent hybridization refers to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions are conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization refers to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions are well known to those of skill in the art and are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See Kanehisa (1984) Nucleic Acids Res. 12:203-215.

As used herein, “biological sample” refers to any sample obtained from a living or viral source or other source of macromolecules and biomolecules, and includes any cell type or tissue of a subject from which nucleic acid or protein or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom. Also included are soil and water samples and other environmental samples, viruses, bacteria, fungi, algae, protozoa and components thereof.

It is understood that aspects and embodiments of the invention described herein include “consisting” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Other objects, advantages and features of the present invention will become apparent from the following specification taken in conjunction with the accompanying drawings.

### B. Methods for Simultaneously Detecting a Target SNP in a Sample

In one aspect, the present disclosure provides for a method for simultaneously detecting one target SNP locus or multiple target SNP loci in a sample, which method comprises: conducting multiplex PCR using a target polynucleotide in a sample as a template and multiple pairs of primers for one target SNP locus or multiple target SNP loci, and analyzing multiple PCR products using capillary electrophoresis, wherein said primers are designed so that the lengths of said PCR products from different SNP loci or from different alleles of the same SNP locus are sufficiently distinguishable from each other in capillary electrophoresis analysis.

The present methods can be used for simultaneously detecting one target SNP locus or multiple target SNP loci using any suitable, or any suitable number of, target polynucleotide(s) as a template in a sample. In some embodiments, the present methods are used to simultaneously detecting one target SNP locus or multiple target SNP loci using a single target polynucleotide in a sample that contains one or multiple SNP loci as a template. In other embodiments, the present methods are used to simultaneously detecting multiple target SNP loci using multiple target polynucleotides in a sample that contain multiple SNP loci as a template.

The present methods can be used for simultaneously detecting any suitable number of target SNP locus or loci. For example, the present methods can be used for simultaneously detecting one SNP locus or at least 2, 3, 4, 5, 6, 7, 8, 9, 10, 11, 12, 13, 14, 15, 16, 17, 18, 19, 20 or more SNP loci in a sample.

The present methods can be used for simultaneously detecting target SNP locus or loci with any suitable number of alleles. For example, the present methods can be used for simultaneously detecting target SNP locus or loci that has or have two or more different alleles.

In some embodiments, the present methods can be used for simultaneously detecting 2-40 different genotypes among 1-20 SNP loci.

In some embodiments, there is no short tandem repeat (STR) or a deletion in the PCR products.

Any suitable primer or primer pairs can be used in the present methods. In some embodiments, there are no other known SNP(s) in the primer sequences. In other embodiments, the annealing temperature(s) for the primer pairs are designed to be used in a single amplification reaction.

In some embodiments, within at least one, some or all of the multiple pairs of primers, one of the primers uses a target SNP as the 3′ end of the primer. In other embodiments, the corresponding SNP allele primer uses the corresponding SNP allele as the 3′ end and comprises at least one additional nucleotide at the 5′ end compared to the 5′ end of the corresponding target SNP primer. In still other embodiments, the corresponding SNP allele primer comprises 2, 3 or more additional nucleotides at the 5′ end compared to the 5′ end of the corresponding target SNP primer. In some embodiments, wherein within the corresponding region, the target SNP primer and the corresponding SNP allele primer can contain at least one different nucleotide. In other embodiments, within the corresponding region, the target SNP primer and the corresponding SNP allele primer can contain at least 2, 3 or more different nucleotides.

Any suitable number of label(s) can be used in the present methods. For example, two or more different labels can be used in the present methods. In another example, a single label can be used in the present methods. Any suitable label can be used in the present methods. In some embodiments, a soluble label or a particle or particulate label can be used in the present methods. Any suitable soluble label can be used. For example, a soluble label can be a colorimetric, a radioactive, an enzymatic, a luminescent or a fluorescent label. Any suitable particle or particulate label can be used. For example, the particle or particulate label can be a colloidal gold label, a latex particle label, a nanoparticle label or a quantum dot label.

The present methods can be used for any suitable purpose. In some embodiments, the present methods can be used for simultaneously detecting one SNP locus or multiple target SNP loci associated with a therapy. For example, the present methods can be used for simultaneously detecting one SNP locus or multiple target SNP loci associated to 5-fluorouracil (5-FU) medication. Any suitable target SNP locus or loci associated to 5-fluorouracil (5-FU) medication can be used. For example, one SNP locus or multiple target SNP loci in target gene(s) encoding protein(s) selected from the group consisting of excision repair cross-complementing rodent repair deficiency, complementation group 2 (ERCC2), dihydropyrimidine dehydrogenase 2A (DPYD*2A), glutathione S-transferase P1 (GSTP1), methylenetetrahydrofolate reductase (NAD(P)H) (MTHFR), orotate phosphoribosyltransferase (OPRT), nitric oxide synthase 3 (NOS3), dihydropyrimidine dehydrogenase 9A (DYPD*9A) and thymidylate synthase (TS) can be simultaneously detected. In another example, one SNP locus or multiple target SNP loci selected from the group consisting of rs13181, rs3918290, rs1695, rs1801133, rs1801019, rs1799983, rs1801265 and rs34743033 can be simultaneously detected.

In some embodiments, the present methods can further comprise conducting a PCR on an internal control polynucleotide. Any suitable internal control polynucleotide can be used. For example, the internal control polynucleotide can comprise a plasmid pcDNA3.1(+).

In some embodiments, the present methods can further comprise conducting multiplex PCR using a positive control target polynucleotide as a template. Any suitable, or any suitable number of, positive control target polynucleotide can be used. For example, the present methods can further comprise conducting multiplex PCR using a single positive control target polynucleotide as a template. In another example, the present methods can further comprise conducting multiplex PCR using at least two positive control target polynucleotides as templates.

In some embodiments, the at least one positive control target polynucleotide can be comprised in a positive control panel that comprises all of the multiple SNP loci and/or their alleles to be detected in a mixture of plasmids. The positive control panel can further comprise an internal control polynucleotide. Any suitable internal control polynucleotide can be used. For example, the internal control polynucleotide can comprise a plasmid pcDNA3.1(+). The positive control for different alleles of the same target SNP locus can be used at any suitable ratio. For example, the positive control panel can comprise at least two different alleles of the same target SNP locus at about 1:1 ratio.

The present methods can be used for simultaneously detecting one SNP locus or multiple target SNP loci on a target polynucleotide from any suitable sample. For example, the present methods can be used for simultaneously detecting one SNP locus or multiple target SNP loci on a target polynucleotide obtained or derived from a biological sample. Any suitable biological sample can be used. For example, the biological sample can be obtained or derived from a human or a non-human mammal. In another example, the biological sample is a whole blood, a plasma, a fresh blood, a blood not containing an anti-coagulate, a urine, a saliva sample, mucosal cells, and cells from a human or a non-human mammal.

### C. Kits and Systems for Simultaneously Detecting a Target SNP in a Sample

In another aspect, the present disclosure provides for a kit or system for simultaneously detecting one target SNP locus or multiple target SNP loci in a sample, which kit or system comprises: 1) multiple pairs of primers for one target SNP locus or multiple target SNP loci; 2) means for conducting multiplex PCR using a target polynucleotide in a sample as a template and said multiple pairs of primers; and 3) means for analyzing multiple PCR products using capillary electrophoresis, wherein said primers are designed so that the lengths of said PCR products from different SNP loci or from different alleles of the same SNP locus are sufficiently distinguishable from each other in capillary electrophoresis analysis.

The present kits can comprise any suitable, or any suitable number of, multiple pairs of primers. In some embodiments, the present kits comprise multiple pairs of primers for simultaneously detecting one SNP locus or at least 2, 3, 4, 5, 6, 7, 8, 9, 10, 11, 12, 13, 14 15, 16, 17, 18, 19 or 20 SNP loci in a sample. In other embodiments, the present kits comprise multiple pairs of primers for simultaneously detecting at least one SNP locus having two or more different alleles. In still other embodiments, the present kits comprise multiple pairs of primers for simultaneously detecting 2-40 different genotypes among 1-20 different SNP loci.

Any suitable primers or primer pairs can be used in the present kits or systems. For example, the present kits or systems can comprise multiple pairs of primers that contain no other known SNP(s). In another example, the present kits or systems can comprise multiple pairs of primers that have the annealing temperatures designed to be used in a single amplification reaction.

In some embodiments, within at least one, some or all of the multiple pairs of primers, one of the primers uses a target SNP as the 3′ end of the primer. In other embodiments, the corresponding SNP allele primer can use the corresponding SNP allele as the 3′ end and can comprise at least one additional nucleotide at the 5′ end compared to the 5′ end of the corresponding target SNP primer. In still other embodiments, the corresponding SNP allele primer can comprise 2, 3 or more additional nucleotides at the 5′ end compared to the 5′ end of the corresponding target SNP primer. In some embodiments, within the corresponding region, the target SNP primer and the corresponding SNP allele primer can contain at least one different nucleotide. In other embodiments, within the corresponding region, the target SNP primer and the corresponding SNP allele primer can contain at least one or more different nucleotides.

Any suitable number of label(s) can be used in the present kits or systems. For example, two or more different labels can be used in the present kits or systems. In another example, a single label can be used in the present kits or systems. Any suitable label can be used in the present kits or systems. In some embodiments, a soluble label or a particle or particulate label can be used in the present kits. Any suitable soluble label can be used. For example, a soluble label can be a colorimetric, a radioactive, an enzymatic, a luminescent or a fluorescent label. Any suitable particle or particulate label can be used. For example, the particle or particulate label can be a colloidal gold label, a latex particle label, a nanoparticle label or a quantum dot label.

The present kits or systems can be used for any suitable purpose. In some embodiments, the present kits or systems can be used for simultaneously detecting one SNP locus or multiple target SNP loci associated with a therapy. For example, the present kits or systems can be used for simultaneously detecting one SNP locus or multiple target SNP loci associated with 5-fluorouracil (5-FU) medication. Any suitable target SNP locus or loci associated with 5-fluorouracil (5-FU) medication can be used. For example, one SNP locus or multiple target SNP loci in target gene(s) encoding protein(s) selected from the group consisting of excision repair cross-complementing rodent repair deficiency, complementation group 2 (ERCC2), dihydropyrimidine dehydrogenase 2A (DPYD*2A), glutathione S-transferase P1 (GSTP1), methylenetetrahydrofolate reductase (NAD(P)H) (MTHFR), orotate phosphoribosyltransferase (OPRT), nitric oxide synthase 3 (NOS3), dihydropyrimidine dehydrogenase 9A (DYPD*9A) and thymidylate synthase (TS) can be simultaneously detected. In another example, one SNP locus or multiple target SNP loci selected from the group consisting of rs13181, rs3918290, rs1695, rs1801133, rs1801019, rs1799983, rs1801265 and rs34743033 can be simultaneously detected.

In some embodiments, the present kits or systems can further comprise an internal control polynucleotide and/or a pair of primers for conducting PCR using the internal control polynucleotide as a template. Any suitable internal control polynucleotide can be used. For example, the internal control polynucleotide can comprise a plasmid pcDNA3.1(+). Any suitable primers or primer pairs can be used. For example, the multiple pairs of primers can comprise, consist essentially of or consist essentially of polynucleotide sequences set forth in Table 5.

In some embodiments, the present kits or systems can further comprise at least one or two positive control target polynucleotide(s) and/or at least one pair of primers for conducting multiplex PCR using the internal control polynucleotide as a template. The at least one positive control target polynucleotide can be stored and/or used in any suitable format. For example, the at least one or two positive control target polynucleotides can be comprised in a positive control panel that comprises all of the multiple SNP loci and their alleles to be detected in a mixture of plasmids. In some embodiments, the positive control panel can further comprise an internal control polynucleotide. Any suitable internal control polynucleotide can be used. For example, the internal control polynucleotide can comprise a plasmid pcDNA3.1(+). The positive control for different alleles of the same target SNP locus can be used at suitable ratio. For example, the positive control panel can comprise at least two different alleles of the same target SNP locus at about 1:1 ratio.

In some embodiments, the present kits or systems can further comprise means for obtaining and/or preparing the target polynucleotide(s).

The present kits or systems can comprise any suitable means for conducting multiplex PCR. For example, the means for conducting multiplex PCR can comprise reagent(s) and/or instrument(s) for conducting multiplex PCR. Any suitable reagents for conducting multiplex PCR can be comprised in the present kits or systems. For example, the reagents for conducting multiplex PCR can comprise PCR buffer and a polynucleotide polymerase.

The present kits or systems can comprise any suitable means for analyzing multiple PCR products. For example, the means for analyzing multiple PCR products can comprise reagent(s) and/or instrument(s) for conducting capillary electrophoresis.

### D. Polynucleotides and Primer Compositions

In yet another aspect, the present disclosure provides for an isolated polynucleotide which comprises a polynucleotide sequence that exhibits at least 70%, 75%, 80%, 85%, 90%, 95%, 99% or 100% identity to any of the ERCC2, DPYD, GSTP1, MTHFR, OPRT, NOS3, DYPD, TS and pcDNA3.1(+) polynucleotide sequences set forth in Table 5, wherein said polynucleotide does not comprise a wild-type, full length ERCC2, DPYD, GSTP1, MTHFR, OPRT, NOS3, DYPD, TS and pcDNA3.1(+) polynucleotide sequence from which said polynucleotide is derived.

In some embodiments, the isolated polynucleotide hybridizes to any of the ERCC2, DPYD, GSTP1, MTHFR, OPRT, NOS3, DYPD, TS and PcDNA3.1(+) polynucleotide sequences set forth in Table 5 under moderately or highly stringent conditions.

In some embodiments, the isolated polynucleotide comprises any of the ERCC2, DPYD, GSTP1, MTHFR, OPRT, NOS3, DYPD, TS and PcDNA3.1(+) polynucleotide sequences set forth in Table 5. In other embodiments, the isolated polynucleotide consists essentially of any of the ERCC2, DPYD, GSTP1, MTHFR, OPRT, NOS3, DYPD, TS and PcDNA3.1(+) polynucleotide sequences set forth in Table 5. In still other embodiments, the isolated polynucleotide consists of any of the ERCC2, DPYD, GSTP1, MTHFR, OPRT, NOS3, DYPD, TS and pcDNA3.1(+) polynucleotide sequences set forth in Table 5. In yet other embodiments, the isolated polynucleotide is complementary or substantially complementary to any of the ERCC2, DPYD, GSTP1, MTHFR, OPRT, NOS3, DYPD, TS and PcDNA3.1(+) polynucleotide sequences set forth in Table 5.

In yet another aspect, the present disclosure provides for a primer composition, which primer composition comprises, consists essentially of or consists of any of the primer pairs set forth in Table 5. In some embodiments, the primer composition comprises, consists essentially of or consists of at least 2, 3, 4, 5, 6, 7, 8, 9, 10, 11, 12, 13, 14, 15, and 16 of the primer pairs set forth in Table 5.

The polynucleotides or the primers can be made using any suitable methods. For example, the polynucleotides or the primers can be made using chemical synthesis, recombinant production or a combination thereof. See e.g., Molecular Cloning, A Laboratory Manual, Second Edition, Cold Spring Harbor Laboratory Press (1989), Current Protocols in Molecular Biology, John Wiley & Sons (1987-1997) or the like.

### E. Exemplary Embodiments

In some embodiments, the present disclosure relates to methods and compositions for detecting multiple SNPs in a sample based on multiplex PCR and CE separation of DNA fragment length size, and the uses of the methods and compositions to detect 8 SNPs for 5-FU medication guide.

The multiple SNPs assay technique can include specific primer design for multiple SNP loci detection and a reference gene and positive control preparation.

In some embodiments, an 8 SNP testing kit for 5-FU medication guide and its detection procedure are disclosed here. The kit can simultaneously or synchronously detect 8 SNP loci including DPD rs3918290, DPD rs1801265, GSTP1 rs1695, MTHFR rs1801133, OPRT rs1801019, TYMS rs37473033, NOS3 rs1799983 and ERCC2 rs13181. The kit is comprised of ultrapure water, solution X, 10×PCR buffer, PCR primers, 25 mM magnesium chloride solution, DNA polymerase, and the positive control. PCR primers include the reverse and forward primers of the 8 SNP loci and an internal control gene. The sequence of the primers is disclosed in the embodiment. The test process includes: sample collection; preparation of nucleic acids; PCR amplification with patient nucleic acids as templates; signal separation using capillary electrophoresis; software identification of SNP loci and file reports.

The exemplary advantages of the embodiment are listed below:

1) Multiple SNP Loci Detection: The embodiment can synchronously or simultaneously detect 1-20 SNP loci with 2-40 genotypes.

2) High-accuracy and high-sensitivity: by means of laser-induced fluorescence-PMT, multiplex PCR and CE separation has a very high signal-to-noise ratio that increases sensitivity and reproducibility across samples for more accurate and informative results.

3) High-specificity: With the proprietary specific primer design and high resolution capillary electrophoretic separation, the technique has a specificity up to about >99%.

4) High-throughput: With the capacity to analyze up to 40 gene targets per reaction and 192 samples per run.

5) Internal reaction control: use of a reaction to reduce or avoid false positive and false negative.

6) Use of software to identify different SNP loci and to conduct the data analysis.

7) Cost-effectiveness and time-saving: by lowering PCR expenses and improving efficiency, the multiplex power of multiplex PCR and CE separation technique enables an user to analyze up to 20 SNP Loci (40 genotypes) per sample at a dramatically reduced cost per target gene and resulted in considerable time savings.

In some embodiments, the term “polymerase chain reaction (PCR)” is a biochemical technology in molecular biology to amplify a single or a few copies of a piece of DNA across several orders of magnitude, generating thousands to millions of copies of a particular DNA sequence.

In some embodiments, the term “multiplex PCR” is a modification of polymerase chain reaction in order to synchronously or simultaneously detect multiple gene targets, e.g., up to 40 gene targets. This process can amplify genomic DNA samples with multiple primers and a temperature-mediated DNA polymerase in a thermal cycler.

In some embodiments, “capillary electrophoresis (CE)” is designed to separate species based on their size to charge ratio in the interior of a small capillary filled with an electrolyte.

In some embodiments, “Deoxyribonucleic acid (DNA)” is a molecule that encodes the genetic instructions used in the development and functioning of an organism, e.g., human beings.

In some embodiments, “5-fluorouracil (5-FU)” is a drug that is a pyrimidine analog which is used in the treatment of cancer. It works through irreversible inhibition of thymidylate synthase and belongs to the family of drugs of anti-metabolites.

In some embodiments, “primer” is a strand of nucleic acid that serves as a starting point for polynucleotide, e.g., DNA, synthesis.

In some embodiments, “primer pool” is the mix of reverse and forward primers of the target SNP loci, e.g., the 8 target SNP loci for monitoring the 5-FU treatment, and/or the reverse and forward primers of a PCR reaction internal control.

In some embodiments, the use of the word “cloning” refers to the fact that the method involves the replication of a single polynucleotide, e.g., a single DNA molecule, starting from a single living cell to generate a large population of cells containing identical DNA molecules.

In some embodiments, the use of the word “multiplex assay” is a type of assay that simultaneously measures multiple analytes in a single run/cycle of the assay. It is distinguished from procedures that measure one analyte at a time.

In some embodiments, “5-FU Panel” refers all the genes including SNP alleles and an internal control in the 5-FU medication kit.

In some embodiments, “Solution X” refers a solution including deoxynucleotide triphosphates and universal amplification primers. The forward universal primer sequence is AGGTGACACTATAGAATA; the reverse universal primer sequence is GTACGACTCACTATAGGGA. The forward universal primers are labeled with fluorescence.

1. Multi-SNP Loci Detection Technique

In some embodiments, the present embodiment relates to a multiplex SNP loci detection technique, which enables to synchronously or simultaneously detect up to 20 SNP loci with 40 genotypes. The technique is based on multiplex PCR and capillary electrophoresis.

a. Specific Primer Design

In some embodiments, two primers were designed according to the target gene sequence of plus and minus strands. The preferred conditions of primer sequences are: there is no short tandem repeat (STR) or deletions in the amplification product; there are no other known SNP(s) in the primer sequences; the Tm value of the designed primers is carefully considered.

1.1 SNP primer design: In reference to the NCBI specific gene sequence, design the SNP primer by using the target SNP as the 3′ end of the primer. The SNP primer may be designed according to the sequence of plus and minus strands.

1.2 The SNP corresponding allele primer design: changing the 3′ end of the SNP primer sequences with the corresponding nucleotide of the SNP (wild-type), and then extending the 5′ end of the SNP corresponding allele primer several bases for later CE separation.

1.3 Artificially mismatch≧1 nucleotide (s) of the two primers mentioned above to increase specificity.

1.4 According to the required length of PCR product, the other end (upstream/downstream) primers and internal control primers are designed. The fragment length of gene targets of an exemplary 5-FU panel is showed in Table 1.

TABLE 1
The Fragment Length of an Exemplary 5-FU Panel

| Gene | SNP/Control | Genotype | Labeled | Fragment Size |
|---|---|---|---|---|
| ERCC2 | rs13181 | C type | ERCC2 C | 151 |
| | | A type | ERCC2 A | 156 |
| DPYD | rs3918290 | C type | DPYD 1C | 140 |
| | | T type | DPYD 1T | 145 |
| GSTP1 | rs1695 | G type | GSTP1 G | 167 |
| | | A type | GSTP1 A | 172 |
| MTHFR | rs1801133 | C type | MTHFR C | 194 |
| | | T type | MTHFR T | 199 |
| OPRT | rs1801019 | G type | OPRT G | 205 |
| | | C type | OPRT C | 210 |
| NOS3 | rs1799983 | G type | NOS3 G | 177 |
| | | T type | NOS3 T | 182 |
| DYPD | rs1801265 | C type | DYPD 2C | 232 |
| | | T type | DYPD 2T | 237 |
| TS | rs34743033 | 2 Repeats | TS 2rpts | 188 |
| | | 3 Repeats | TS 3rpts | 216 |

| | | | |
|---|---|---|---|
| pcDNA3.1(+) | Reaction Control | DNA Ctl | 225 |

b. Positive Control Preparation

The positive control is prepared by cloning the related SNP allele fragments into plasmids. After quantitating plasmids, a plasmid pool is made by adjusting the plasmids of two alleles of a SNP at about 1:1 or 1:1 ratio to analog heterozygous alleles and then mixing all the related plasmids and the internal control pcDNA3.1 in one tube.

2. A Multiplex SNP Testing Kit for 5-FU Medication Guide and Detection Procedure

a. Multiplex SNP Testing Kit for 5-FU Medication Guide” Comprise the Following Components:

### 1) 5-FU Panel Primer Mix

### 2) Solution X

### 3) 10×PCR Buffer

### 4) 25 mM MgCl₂

### 5) Taq DNA Polymerase

### 6) 5-FU Positive Control

### 7) Ultrapure H₂O

b. Detection Procedure

### 2.1 Collecting Samples

Specimens from patients' mouth swabs or blood samples are collected.

### 2.2 Preparation of Nucleic Acids

Alkaline Lysis Method is used for human DNA extraction from mouth swab sample. Any commercial human DNA extraction kit that can extract DNA from blood/mouth swab will be applicable for the procedure.

3. PCR Amplification with Patient Nucleic Acids or 5-FU Positive Control as Templates.

TABLE 2
20 μL PCR Reaction System of 5-FU Panel

| PCR reagents | Volume/well |
|---|---|
| 10X PCR buffer | 2 μL |
| 25 mM MgCl₂ | 4 μL |
| 5-FU Primer Mix | 2 μL |
| Taq DNA polymerase | 1 μL |
| Solution X | 2 μL |
| 5-FU Positive control/Sample | 5 μL |
| Ultrapure H2O | 4 μL |

TABLE 3
PCR reaction condition

| Reaction steps | Temperature, time | Cycles |
|---|---|---|
| 1. Initialization step | 94° C., 60 s | 35 cycles |
| 2. Denaturation step | 94° C., 30 s | |
| 3. Annealing step | 60° C., 30 s | |
| 4. Elongation step | 70° C., 60 s | |
| 5. Final elongation | 70° C., 60 s | |
| 6. Final hold | 4° C., ∞ | |

### 4. Capillary Electrophoresis Analysis for Fragment Separation.

### a) Prepare CE Loading Samples (See Table 3).

TABLE 4
CE loading sample

| CE Component | Quantity per Reaction (μL) | |
|---|---|---|
| Sample loading solution | 38.7 | μL |
| DNA size standard 400 | 0.3 | μL |
| PCR product | 1 | μL |
| Mineral oil | 1 | drop |

1) DNA Size Standard-400 with Mineral Oil (PN 608098, Beckman Coulter)

### 2) GenomeLab Sample Loading Solution (PN 608082, Beckman Coulter)

### b) CE Separation of the DNA Fragment Signals in the PCR Product

The present invention is further illustrated by the following exemplary embodiments:
1. A multiplex SNP assay technique, an 8 SNP testing kit for 5-FU medication guide and its detection procedure.
2. The multiplex SNP assay technique of embodiment 1, wherein is based on multiplex PCR and capillary electrophoresis technique.
3. The multiplex SNP assay technique of embodiment 1, wherein the technique enables to synchronously detect multiple SNP alleles (up to 15 SNP loci with 30 genotypes).
4. The multiplex SNP assay technique of embodiment 1, wherein identifying SNP alleles based on the fragment length showed on the graphs of capillary electrophoresis.
5. The multiplex SNP assay technique of embodiment 1, wherein the specific primer design method enables to detect multiple SNPs and its corresponding alleles.
6. The specific primer design method of embodiment 5, wherein design the SNP primer by using the target SNP as the 3′ end of the primer.
7. The specific primer design method of embodiment 5, wherein the SNP corresponding allele primers are designed by changing the 3′ end of the SNP primer sequences with the corresponding nucleotide of the SNP (wild-type), and then extending the 5′ end of the SNP corresponding allele primer ≧3 bases for later CE separation.
8. The specific primer design method of embodiment 6 and/or 7, wherein artificially mismatch ≧1 nucleotide (s) of the SNP primer and the SNP corresponding allele primer to increase specificity.
9. The specific primer design method of any of embodiments 5-8, wherein the primer sequences are disclosed in Table 5.

TABLE 5
The Primer Sequences of 5-FU Panel

| | SNP/ | Geno- | |
|---|---|---|---|
| Gene | Control | type | Primer Sequence |
| ERCC2 | rs13181 | For- | CCAGGGCCAGGCAAGAC |
| | | ward | |
| | | C | CTAGAATCAGAGGAGACGCTGC |
| | | type | |
| | | A | ACTGGCTAGAATCAGAGGAG |
| | | type | ACGCTGA |
| DPYD | rs3918290 | For- | TTATAAGCCTATGAATTGGAT |
| | | ward | |
| | | C | GGCTGACTTTCCAGACAACG |
| | | type | |
| | | T | CTAAAGGCTGACTTTCCAGACA |
| | | type | ACA |
| GSTP1 | rs1695 | For- | CACGCGGCCTGCTCCCCTC |
| | | ward | |
| | | G | TTGGTGTAGATGAGGGAGAC |
| | | type | |
| | | A | CATAGTTGGTGTAGATGAGGGA |
| | | type | GAT |
| MTHFR | rs1801133 | For- | GGGCTCTCCTGGGCCCCTCA |
| | | ward | |
| | | C | GAGAAGGTGTCTGCGGGAGC |
| | | type | |
| | | T | CGAAGGAGAAGGTGTCTGCGGG |
| | | type | AGT |
| OPRT | rs1801019 | For- | CAGGCGCACGGGATCCGCCT |
| | | ward | |
| | | G | CTTTATAGAAAGGGGAGAAC |
| | | type | |
| | | C | ACTTCCTTTATAGAAAGGGGAG |
| | | type | AAG |
| NOS3 | rs1799983 | For- | TCTTGAGAGGCTCAGGGATG |
| | | ward | |
| | | G | TGCAGGCCCCAGATGAG |
| | | type | |
| | | T | GCTGCTGCAGGCCCCAGATGAT |
| | | type | |
| DYPD | rs1801265 | For- | CGGCTGTACTTTAATACCTTAT |
| | | ward | TTC |
| | | C | AACACAAACTCATGCAACTCTGC |
| | | type | |
| | | T | CCTCGAACACAAACTCATGCAA |
| | | type | CTCTGT |
| TS | rs34743033 | For- | GCGGAAGGGGTCCTGCCA |
| | | ward | |
| | | Re- | CGTCCCGCTCCTGTGCG |
| | | verse | |
| pcDNA | Reaction | For- | CAGACAATCGGCTGCTCTGA |
| | | ward | |
| 3.1(+) | Control | Re- | CTTCCCGCTTCAGTGACAAC |
| | | verse | |

10. The 8 SNP testing kit for 5-FU medication guide of embodiment 1, wherein the kit can synchronously detect 8 SNP loci including rs13181, rs3918290, rs1695, rs1801265, rs1801133, rs1801019, rs1799983 and rs37473033 and an internal control.
11. The 8 SNP testing kit for 5-FU medication guide of embodiment 1, wherein the internal control is a plasmid pcDNA3.1(+).
12. The internal control of embodiment 11, the plasmid pcDNA 3.1(+) is used to confirm that the PCR reaction is processed successfully.
13. The method and kit of embodiment 1, wherein a mix of plasmids (plasmid pool) with cloned SNP alleles and pcDNA3.1 (+) is used as positive control of the panel.
14. The method and kit of embodiment 1 and/or 13, wherein the plasmid pool is made by adjusting the plasmids of a SNP two alleles at 1:1 ratio to analog heterozygous alleles.
15. The kit of embodiment 1, wherein the components of the kit are as follows:

### 1) 5-FU Panel Primer Mix

### 2) Solution X

### 3) 10×PCR Buffer

### 4) 25 mM MgCl₂

### 5) Taq DNA Polymerase

### 6) 5-FU Positive Control

### 7) Ultrapure H₂O

16. The kit of embodiment 1, wherein the extracted DNA from human mouth swab/blood samples can be used as template in the kit.

### F. Examples

### Example 1

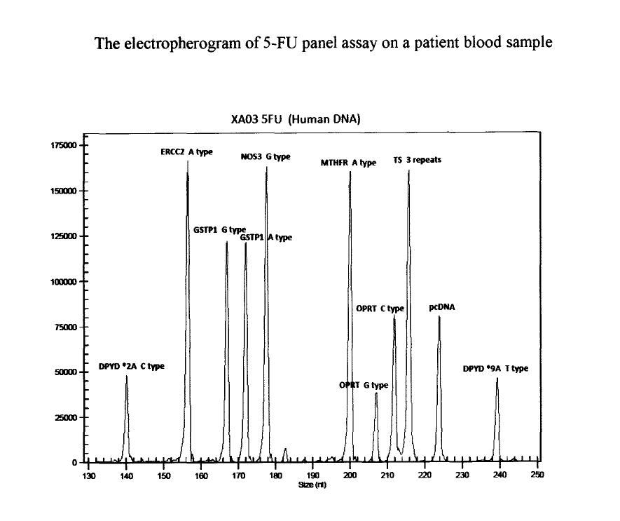

After sample collection and preparation of nucleic acids, PCR amplification with patient nucleic acids as templates and fragment separation by capillary electrophoresis (CE) was conducted. The electrophoresis graph and analyzed results are shown in FIG. 1.

The electropherogram in FIG. 1 shows the analysis result of the 5-FU panel using a patient blood sample as template. The 5-FU panel is able to simultaneously analyze 8 SNP genotypes associated with the toxicity/ADR, efficacy and prognosis of 5-FU and a reference (internal PCR control). The alleles of the patent are: ERCC2 AA, DYPD CC (rs3918290), GSTP1 GA, NOS3 GG, TS 3 repeats, MTHFR AA, OPRT GC, and DYPD TT (rs1801265).

Citation of the above publications or documents is not intended as an admission that any of the foregoing is pertinent prior art, nor does it constitute any admission as to the contents or date of these publications or documents.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

1. A method for simultaneously detecting one target SNP locus or multiple target SNP loci in a sample, which method comprises:
conducting multiplex PCR using a target polynucleotide or multiple target polynucleotides in a sample as a template or templates and multiple pairs of primers for one SNP locus or multiple target SNP loci, and analyzing multiple PCR products using capillary electrophoresis,
wherein said primers are designed so that the lengths of said PCR products from different SNP loci or from different alleles of the same SNP locus are sufficiently distinguishable from each other in capillary electrophoresis analysis.

2-3. (canceled)

4. The method of claim 1, which is used for simultaneously detecting one SNP locus or at least 2, 3, 4, 5, 6, 7, 8, 9, 10, 11, 12, 13, 14, 15, 16, 17, 18, 19, 20 or more SNP loci in a sample.

5-8. (canceled)

9. The method of claim 1, wherein the annealing temperatures for the primer pairs are designed to be used in a single amplification reaction.

10. The method of claim 1, wherein within at least one, some or all of the multiple pairs of primers, one of the primers uses a target SNP as the 3′ end of the primer.

11. The method of claim 10, wherein the corresponding SNP allele primer uses the corresponding SNP allele as the 3′ end and comprises at least one additional nucleotide at the 5′ end compared to the 5′ end of the corresponding target SNP primer.

12. (canceled)

13. The method of claim 11, wherein within the corresponding region, the target SNP primer and the corresponding SNP allele primer contain at least one or more different nucleotides.

14. (canceled)

15. The method of claim 1, wherein a single label or more different labels are used.

16. (canceled)

17. The method of claim 15, wherein the label is a soluble label or a particle or particulate label.

18. The method of claim 17, wherein the soluble label is a colorimetric, a radioactive, an enzymatic, a luminescent or a fluorescent label.

19. The method of claim 17, wherein the particle or particulate label is a colloidal gold label, a latex particle label, a nanoparticle label or a quantum dot label.

20. The method of claim 1, which is used for simultaneously detecting one target SNP locus or multiple target SNP loci associated with a therapy.

21-23. (canceled)

24. The method of claim 1, which further comprises conducting a PCR on an internal control polynucleotide and/or further comprises conducting multiplex PCR using a positive control target polynucleotide as a template.

25-27. (canceled)

28. The method of claim 24, wherein the positive control target polynucleotide is comprised in a positive control panel that comprises all of the multiple SNP loci and/or their alleles to be detected in a mixture of plasmids.

29-30. (canceled)

31. The method of claim 28, wherein the positive control panel comprises at least two different alleles of the same target SNP locus at about 1:1 ratio.

32. The method of claim 1, wherein the target polynucleotide is obtained or derived from a biological sample.

33-34. (canceled)

35. A kit or system for simultaneously detecting one target SNP locus or multiple target SNP loci in a sample, which kit or system comprises:
1) multiple pairs of primers for one target SNP locus or multiple target SNP loci;
2) means for conducting multiplex PCR using a target polynucleotide in a sample as a template and said multiple pairs of primers; and
3) means for analyzing multiple PCR products using capillary electrophoresis,
wherein said primers are designed so that the lengths of said PCR products from different SNP loci or from different alleles of the same SNP locus are sufficiently distinguishable from each other in capillary electrophoresis analysis.

36-37. (canceled)

38. The kit or system of claim 35, which comprises multiple pairs of primers for simultaneously detecting 2-40 different genotypes among 1-20 different SNP loci.

39-40. (canceled)

41. The kit or system of claim 35, wherein within at least one, some or all of the multiple pairs of primers, one of the primers uses a target SNP as the 3′ end of the primer.

42. The kit or system of claim 41, wherein the corresponding SNP allele primer uses the corresponding SNP allele as the 3′ end and comprises at least one additional nucleotide at the 5′ end compared to the 5′ end of the corresponding target SNP primer.

43. (canceled)

44. The kit or system of claim 42, wherein within the corresponding region, the target SNP primer and the corresponding SNP allele primer contain at least one or more different nucleotides.

45-74. (canceled)


SEQUENCE LISTING (27 sequences)

SEQ ID NO: 1 - DNA, 18 bp, artificial sequence
  forward universal primer in Solution X
aggtgacactatagaata

SEQ ID NO: 2 - DNA, 19 bp, artificial sequence
  reverse universal promer in Solution X
gtacgactcactataggga

SEQ ID NO: 3 - DNA, 17 bp, artificial sequence
  forward primer C/A type of rs12181
ccagggccaggcaagac

SEQ ID NO: 4 - DNA, 22 bp, artificial sequence
  reverse primer for C type of rs13181
ctagaatcagaggagacgctgc

SEQ ID NO: 5 - DNA, 27 bp, artificial sequence
  reverse primer for A type of rs13181
actggctagaatcagaggagacgctga

SEQ ID NO: 6 - DNA, 21 bp, artificial sequence
  forward primer for C/T type of rs3918290
ttataagcctatgaattggat

SEQ ID NO: 7 - DNA, 20 bp, artificial sequence
  reverse primer for C type of rs3918290
ggctgactttccagacaacg

SEQ ID NO: 8 - DNA, 25 bp, Artificial sequence
  reverse primer for T type of rs3918290
ctaaaggctgactttccagacaaca

SEQ ID NO: 9 - DNA, 19 bp, artificial sequence
  forward primer for G/A type of rs1695
cacgcggcctgctcccctc

SEQ ID NO: 10 - DNA, 20 bp, artificial sequence
  reverse primer for G type of rs1695
ttggtgtagatgagggagac

SEQ ID NO: 11 - DNA, 25 bp, artificial sequence
  reverse primer for A type of rs1695
catagttggtgtagatgagggagat

SEQ ID NO: 12 - DNA, 20 bp, artificial sequence
  forward primer for C/T type of rs1801133
gggctctcctgggcccctca

SEQ ID NO: 13 - DNA, 20 bp, artificial sequence
  reverse primer for C type of rs1801133
gagaaggtgtctgcgggagc

SEQ ID NO: 14 - DNA, 25 bp, artificial sequence
  reverse primer for T type of rs1801133
cgaaggagaaggtgtctgcgggagt

SEQ ID NO: 15 - DNA, 20 bp, artificial sequence
  forward primer for G/C type of rs1801019
caggcgcacgggatccgcct

SEQ ID NO: 16 - DNA, 20 bp, artificial sequence
  reverse primer for G type of rs1801019
ctttatagaaaggggagaac

SEQ ID NO: 17 - DNA, 25 bp, artificial sequence
  reverse primer for C type of rs1801019
acttcctttatagaaaggggagaag

SEQ ID NO: 18 - DNA, 20 bp, artificial sequence
  forward primer for G/T type of rs1799983
tcttgagaggctcagggatg

SEQ ID NO: 19 - DNA, 17 bp, artificial sequence
  reverse primer for G type of rs1799983
tgcaggccccagatgag

SEQ ID NO: 20 - DNA, 22 bp, artificial sequence
  reverse primer for T type of rs1799983
gctgctgcaggccccagatgat

SEQ ID NO: 21 - DNA, 25 bp, artificial sequence
  forward primer for C/T type of rs1801265
cggctgtactttaataccttatttc

SEQ ID NO: 22 - DNA, 23 bp, artificial sequence
  reverse primer for C type of rs1801265
aacacaaactcatgcaactctgc

SEQ ID NO: 23 - DNA, 28 bp, artificial sequence
  reverse primer for T type of rs1801265
cctcgaacacaaactcatgcaactctgt

SEQ ID NO: 24 - DNA, 18 bp, artificial sequence
  forward primer for rs34743033
gcggaaggggtcctgcca

SEQ ID NO: 25 - DNA, 17 bp, artificial sequence
  reverse primer for rs34743033
cgtcccgctcctgtgcg

SEQ ID NO: 26 - DNA, 20 bp, artificial sequence
  forward primer for reaction control
cagacaatcggctgctctga

SEQ ID NO: 27 - DNA, 20 bp, artificial sequence
  reverse primer for reaction control
cttcccgcttcagtgacaac